(19) United States
(12) Patent Application Publication
(10) Pub. No.: US 2023/0101030 A1
(43) Pub. Date: Mar. 30, 2023

(54) Shape Sensing Reference Frame

(71) Applicant: Bard Access Systems, Inc., Salt Lake City, UT (US)

(72) Inventors: Anthony K. Misener, Bountiful, UT (US); Steffan Sowards, Salt Lake City, UT (US); William Robert McLaughlin, Bountiful, UT (US)

(21) Appl. No.: 17/955,019

(22) Filed: Sep. 28, 2022

Related U.S. Application Data
(60) Provisional application No. 63/250,727, filed on Sep. 30, 2021.

(51) Int. Cl. A61B 34/20

(52) U.S. Cl. CPC A61B 34/20; A61B 2034/2061

(57) ABSTRACT

Disclosed herein is a system and method directed to detecting placement of a medical device within a patient body, where the system includes a medical device including an optical fiber having core fibers. Each of the one or more core fibers includes a plurality of sensors that can be configured to reflect a light signal having an altered characteristic due to strain experienced by the optical fiber. The system further includes logic that can be configured to determine a 3D shape of the medical device in accordance with the strain of the optical fiber. The logic can further be configured to (i) define a reference frame for the 3D shape, and (ii) render an image of the 3D shape on a display of the system in accordance with the reference frame.

### PRIORITY

This application claims the benefit of priority to U.S. Provisional Application No. 63/250,727, filed Sep. 30, 2021, which is incorporated by reference in its entirety into this application.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

Disclosed herein is a fiber optic shape sensing system and methods performed thereby where the system is configured to display an image of three-dimensional shape of a medical device using optical fiber technology. Further, the system is configured to define a reference frame for the three-dimensional shape to enable to the clinician to view an image the three-dimensional shape according to defined orientations of the three-dimensional shape.

### SUMMARY

Briefly summarized, disclosed herein is a medical device system for detecting placement of a medical device within a patient body. The system includes the medical device including an optical fiber having one or more of core fibers, each of the one or more core fibers including a plurality of sensors distributed along a longitudinal length of a corresponding core fiber and each sensor of the plurality of sensors being configured to (i) reflect a light signal of a different spectral width based on received incident light, and (ii) change a characteristic of the reflected light signal based on strain experienced by the optical fiber. The system further includes a console including one or more processors and a non-transitory computer-readable medium having stored thereon logic, when executed by the one or more processors, causes operations of the system. The operations include (i) providing an incident light signal to the optical fiber, (ii) receiving reflected light signals of different spectral widths of the incident light by one or more of the plurality of sensors, (iii) processing the reflected light signals associated with the one or more of core fibers to determine a three-dimensional (3D) shape of the optical fiber, (iv) defining a reference frame for displaying an image of the 3D shape, (v) orienting the 3D shape within the reference frame, and (vi) rendering an image of the 3D shape on a display of the system in accordance with the reference frame.

In some embodiments, orienting the 3D shape includes defining a reference plane according to a portion of the 3D shape, fixing the 3D shape to the reference plane, and orienting the reference plane with respect to the reference frame. The portion of the 3D shape may include three or more points disposed along the 3D shape, and the three or more points may be equidistant from the reference plane.

The system may further include a guide having a lumen extending along a straight section of the guide, where in use, the optical fiber is inserted within the lumen, and the operations further include calibrating the optical fiber in accordance with the straight section. In some embodiments, the guide is inserted within the patient.

The operations may further include orienting the reference plane with respect to the reference frame so that a front view image of the 3D shape according to the reference frame is aligned with a front view of the patient and the operations further include fixing the reference plane with respect to the reference frame.

In some embodiments, the operations further include comparing a curved portion of the 3D shape with a curved shape stored in memory, and as a result of the comparison, identifying the three or more points from the curved portion to define the reference plane when the curved portion of the 3D shape is consistent with the curved shape stored in memory. In use, the curved portion of the 3D shape may be disposed along a basilic vein, a subclavian vein, an innominate vein, or a superior vena cava, of the patient.

The system may include a plurality of curved shapes stored in memory pertaining to a plurality of different insertion sites for the medical device, and the operations may further include (i) receiving input from a clinician defining an insertion site for the medical device, (ii) selecting a curved shape from the plurality of curved shapes, the selected curved shape pertaining to the defined insertion site, and (iii) comparing the curved portion of the 3D shape with the selected curved shape. The clinician input may further define the insertion site as located on a right side or a left side of the patient.

The system may further include a reference guide coupled with the medical device, where the curved portion of the 3D shape is disposed along a pathway of the reference guide, and an orientation of the reference guide with respect to the patient defines an orientation of the 3D shape with respect to the patient. In use, the reference guide may be displaced between a first guide orientation and a second guide orientation with respect to the patient to move the 3D shape between a first 3D shape orientation and a second 3D shape orientation with respect to the patient.

The system may be coupled with a patient imaging system, and the operations may further include receiving image data from the imaging system, and rendering an image of the patient on the display along with the image of the 3D shape.

The medical device may be one of an introducer wire, a guidewire, a stylet, a stylet within a needle, a needle with the optical fiber inlayed into a cannula of the needle or a catheter with the optical fiber inlayed into one or more walls of the catheter.

Also disclosed herein is a method for detecting placement of a medical device within a patient body. The method includes providing an incident light signal to an optical fiber included within the medical device, wherein the optical fiber includes a one or more of core fibers, each of the one or more of core fibers including a plurality of reflective gratings distributed along a longitudinal length of a corresponding core fiber and each of the plurality of reflective gratings being configured to (i) reflect a light signal of a different spectral width based on received incident light, and (ii) change a characteristic of the reflected light signal based on strain experienced by the optical fiber. The method further includes (i) receiving reflected light signals of different spectral widths of the incident light by one or more of the plurality of sensors, (ii) processing the reflected light signals associated with the one or more of core fibers to determine a three-dimensional (3D) shape of the optical fiber, (iii) defining a reference frame for displaying an image of the 3D shape, (iv) orienting the 3D shape within the reference frame, and (v) rendering an image of the 3D shape on a display of a system in accordance with the reference frame.

In some embodiments of the method, orienting the 3D shape includes defining a reference plane according to a portion of the 3D shape, fixing the 3D shape to the reference plane, and orienting the reference plane with respect to the reference frame. The portion of the 3D shape may include three or more points disposed along the 3D shape, and the three or more points may be equidistant from the reference plane.

In some embodiments of the method, the system includes a guide having a lumen extending along a straight section of the guide, and in use, the optical fiber is inserted within the lumen. The method further includes calibrating the optical fiber in accordance with the straight section. The guide may be inserted within the patient.

The method may further include (i) orienting the reference plane with respect to the reference frame so that a front view image of the 3D shape according to the reference frame is aligned with a front view of the patient and (ii) fixing the reference plane with respect to the reference frame.

In some embodiments, the method further includes comparing a curved portion of the 3D shape with a curved shape stored in memory of the system, and as a result of the comparison, identifying the three or more points from the curved portion to define the reference plane when the curved portion of the 3D shape is consistent with the curved shape stored in memory.

In some embodiments of the method, the curved portion of the 3D shape is disposed along the a basilic vein, a subclavian vein, an innominate vein, or a superior vena cava, of the patient. In some embodiments of the method, the system includes a plurality of curved shapes stored in memory pertaining to a plurality of different insertion sites, the method further includes (i) receiving input from a clinician defining an insertion site for the medical device, (ii) selecting a curved shape from the plurality of curved shapes, the selected curved shape pertaining to the defined insertion site, and (iii) comparing the curved portion of the 3D shape with the selected curved shape. The clinician input may further define the insertion site as located on a right side or a left side of the patient.

In some embodiments of the method, (i) the system includes a reference guide coupled with the medical device, (ii) the curved portion of the 3D shape is disposed along a pathway of the reference guide, (iii) an orientation of the reference guide with respect to the patient defines an orientation of the 3D shape with respect to the patient.

The method may further include displacing the reference guide between a first guide orientation and a second guide orientation with respect to the patient to move the 3D shape between a first 3D shape orientation and a second 3D shape orientation with respect to the patient.

In some embodiments of the method, the system is coupled with a patient imaging system, and the method further includes receiving image data from the imaging system, and rendering an image of the patient on the display along with the image of the 3D shape.

These and other features of the concepts provided herein will become more apparent to those of skill in the art in view of the accompanying drawings and following description, which disclose particular embodiments of such concepts in greater detail.

### BRIEF DESCRIPTION OF THE DRAWINGS

Embodiments of the disclosure are illustrated by way of example and not by way of limitation in the figures of the accompanying drawings, in which like references indicate similar elements and in which:

FIG. 1A is an illustrative embodiment of a medical instrument monitoring system including a medical instrument with optic shape sensing and fiber optic-based oximetry capabilities in accordance with some embodiments;

FIG. 1B is an alternative illustrative embodiment of the medical instrument monitoring system 100 in accordance with some embodiments;

FIG. 2 is an exemplary embodiment of a structure of a section of the multi-core optical fiber included within the stylet 120 of FIG. 1A in accordance with some embodiments;

FIG. 3A is a first exemplary embodiment of the stylet of FIG. 1A supporting both an optical and electrical signaling in accordance with some embodiments;

FIG. 3B is a cross sectional view of the stylet of FIG. 3A in accordance with some embodiments;

FIG. 4A is a second exemplary embodiment of the stylet of FIG. 1B in accordance with some embodiments;

FIG. 4B is a cross sectional view of the stylet of FIG. 4A in accordance with some embodiments;

FIG. 5A is an elevation view of a first illustrative embodiment of a catheter including integrated tubing, a diametrically disposed septum, and micro-lumens formed within the tubing and septum in accordance with some embodiments;

FIG. 5B is a perspective view of the first illustrative embodiment of the catheter of FIG. 5A including core fibers installed within the micro-lumens in accordance with some embodiments;

FIGS. 6A-6B are flowcharts of the methods of operations conducted by the medical instrument monitoring system of FIGS. 1A-1B to achieve optic 3D shape sensing in accordance with some embodiments;

FIG. 7A is an exemplary embodiment of the medical instrument monitoring system of FIGS. 1A-1B during operation and insertion of the catheter into a patient in accordance with some embodiments;

FIG. 7B illustrates the 3D shape of FIG. 7A in accordance with a reference frame, in accordance with some embodiments;

FIG. 8 is an exemplary screen shot of image of the 3D shape of FIGS. 7A and 7B, in accordance with some embodiments;

FIG. 9 is a flowchart of a methods of operations conducted by the medical instrument monitoring system of FIGS. 1A-1B to display an image of the 3D shape according to the reference frame, in accordance with some embodiments; and

FIG. 10 illustrates an embodiment of a reference guide for use in defining the reference frame of FIGS. 7A and 7B, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

The phrases “connected to” and “coupled with” refer to any form of interaction between two or more entities, including mechanical, electrical, magnetic, electromagnetic, fluid, and thermal interaction. Two components may be connected to or coupled with each other even though they are not in direct contact with each other. For example, two components may be coupled with each other through an intermediate component.

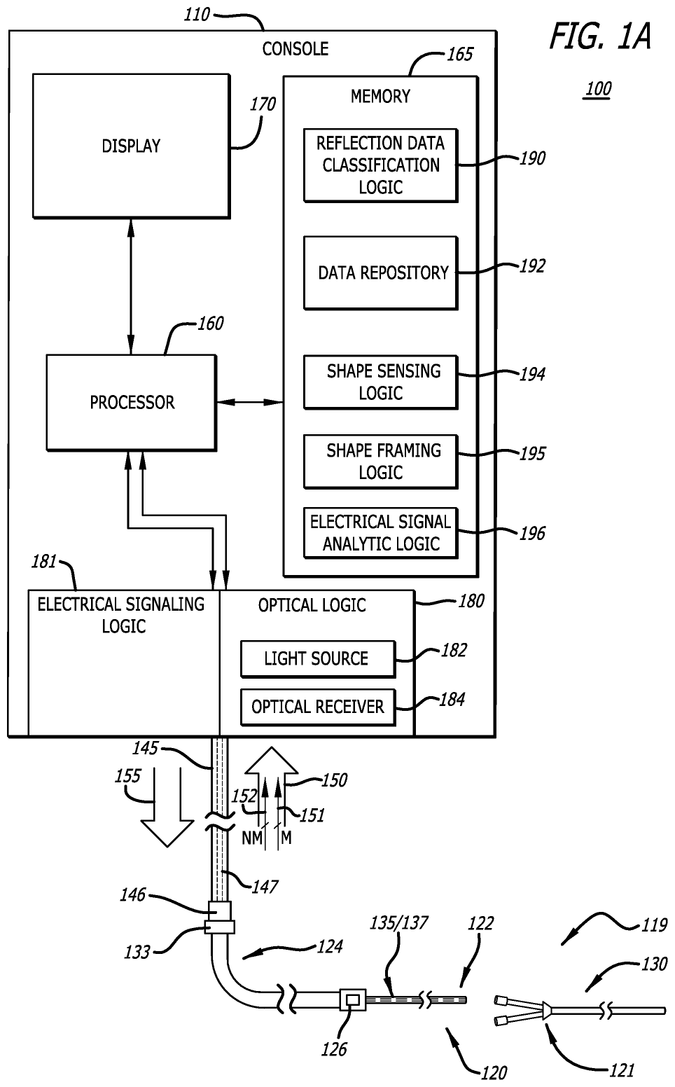

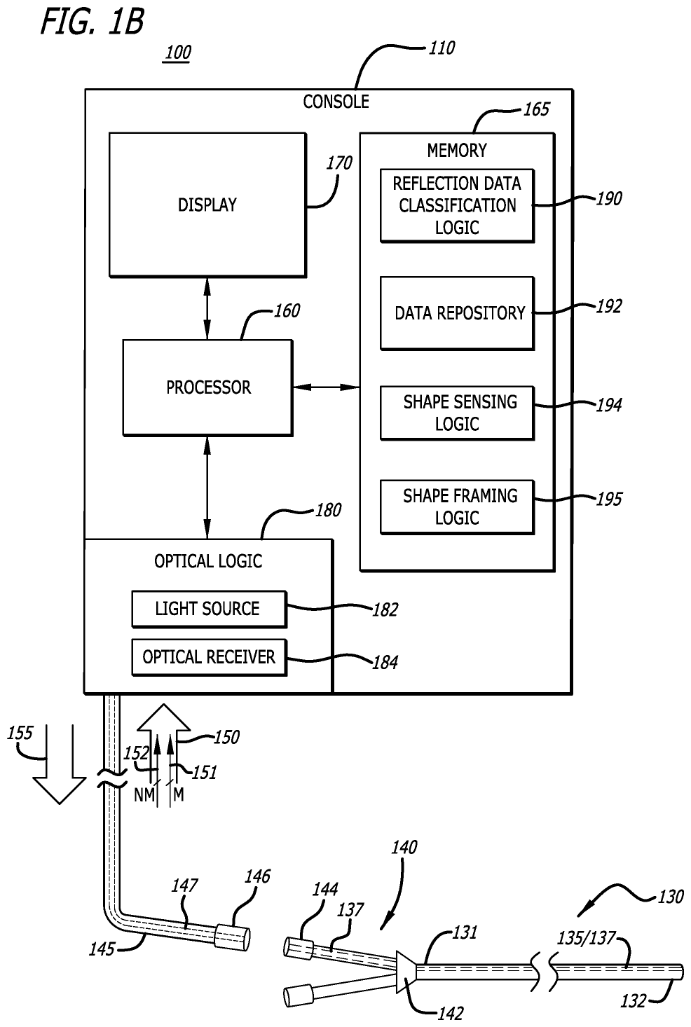

Referring to FIG. 1A, an illustrative embodiment of a medical instrument monitoring system including a medical instrument with optic shape sensing and fiber optic-based oximetry capabilities is shown in accordance with some embodiments. As shown, the system 100 generally includes a console 110 and a stylet assembly 119 communicatively coupled to the console 110. For this embodiment, the stylet assembly 119 includes an elongate probe (e.g., stylet) 120 on its distal end 122 and a console connector 133 on its proximal end 124. The console connector 133 enables the stylet assembly 119 to be operably connected to the console 110 via an interconnect 145 including one or more optical fibers 147 (hereinafter, “optical fiber(s)”) and a conductive medium terminated by a single optical/electric connector 146 (or terminated by dual connectors. Herein, the connector 146 is configured to engage (mate) with the console connector 133 to allow for the propagation of light between the console 110 and the stylet assembly 119 as well as the propagation of electrical signals from the stylet 120 to the console 110.

An exemplary implementation of the console 110 includes a processor 160, a memory 165, a display 170 and optical logic 180, although it is appreciated that the console 110 can take one of a variety of forms and may include additional components (e.g., power supplies, ports, interfaces, etc.) that are not directed to aspects of the disclosure. An illustrative example of the console 110 is illustrated in U.S. Pat. No. 10,992,078, the entire contents of which are incorporated by reference herein. The processor 160, with access to the memory 165 (e.g., non-volatile memory or non-transitory, computer-readable medium), is included to control functionality of the console 110 during operation. As shown, the display 170 may be a liquid crystal diode (LCD) display integrated into the console 110 and employed as a user interface to display information to the clinician, especially during a catheter placement procedure (e.g., cardiac catheterization). In another embodiment, the display 170 may be separate from the console 110. Although not shown, a user interface is configured to provide user control of the console 110.

For both of these embodiments, the content depicted by the display 170 may change according to which mode the stylet 120 is configured to operate: optical, TLS, ECG, or another modality. In TLS mode, the content rendered by the display 170 may constitute a two-dimensional (2D) or three-dimensional (3D) representation of the physical state (e.g., length, shape, form, and/or orientation) of the stylet 120 computed from characteristics of reflected light signals 150 returned to the console 110. The reflected light signals 150 constitute light of a specific spectral width of broadband incident light 155 reflected back to the console 110. According to one embodiment of the disclosure, the reflected light signals 150 may pertain to various discrete portions (e.g., specific spectral widths) of broadband incident light 155 transmitted from and sourced by the optical logic 180, as described below

According to one embodiment of the disclosure, an activation control 126, included on the stylet assembly 119, may be used to set the stylet 120 into a desired operating mode and selectively alter operability of the display 170 by the clinician to assist in medical device placement. For example, based on the modality of the stylet 120, the display 170 of the console 110 can be employed for optical modality-based guidance during catheter advancement through the vasculature or TLS modality to determine the physical state (e.g., length, form, shape, orientation, etc.) of the stylet 120. In one embodiment, information from multiple modes, such as optical, TLS or ECG for example, may be displayed concurrently (e.g., at least partially overlapping in time).

Referring still to FIG. 1A, the optical logic 180 is configured to support operability of the stylet assembly 119 and enable the return of information to the console 110, which may be used to determine the physical state associated with the stylet 120 along with monitored electrical signals such as ECG signaling via an electrical signaling logic 181 that supports receipt and processing of the received electrical signals from the stylet 120 (e.g., ports, analog-to-digital conversion logic, etc.). The physical state of the stylet 120 may be based on changes in characteristics of the reflected light signals 150 received at the console 110 from the stylet 120. The characteristics may include shifts in wavelength caused by strain on certain regions of the core fibers integrated within an optical fiber core 135 positioned within or operating as the stylet 120, as shown below. As discussed herein, the optical fiber core 135 may be comprised of core fibers 137₁-137_{M} (M=1 for a single core, and M≥2 for a multi-core), where the core fibers 137₁-137_{M} may collectively be referred to as core fiber(s) 137. Unless otherwise specified or the instant embodiment requires an alternative interpretation, embodiments discussed herein will refer to a multi-core optical fiber 135. From information associated with the reflected light signals 150, the console 110 may determine (through computation or extrapolation of the wavelength shifts) the physical state of the stylet 120, and also that of a catheter 121 configured to receive the stylet 120.

According to one embodiment of the disclosure, as shown in FIG. 1A, the optical logic 180 may include a light source 182 and an optical receiver 184. The light source 182 is configured to transmit the incident light 155 (e.g., broadband) for propagation over the optical fiber(s) 147 included in the interconnect 145, which are optically connected to the multi-core optical fiber core 135 within the stylet 120. In one embodiment, the light source 182 is a tunable swept laser, although other suitable light sources can also be employed in addition to a laser, including semi-coherent light sources, LED light sources, etc.

The optical receiver 184 is configured to: (i) receive returned optical signals, namely reflected light signals 150 received from optical fiber-based reflective gratings (sensors) fabricated within each core fiber of the multi-core optical fiber 135 deployed within the stylet 120, and (ii) translate the reflected light signals 150 into reflection data (from repository 192), namely data in the form of electrical signals representative of the reflected light signals including wavelength shifts caused by strain. The reflected light signals 150 associated with different spectral widths may include reflected light signals 151 provided from sensors positioned in the center core fiber (reference) of the multi-core optical fiber 135 and reflected light signals 152 provided from sensors positioned in the periphery core fibers of the multi-core optical fiber 135, as described below. Herein, the optical receiver 184 may be implemented as a photodetector, such as a positive-intrinsic-negative “PIN” photodiode, avalanche photodiode, or the like.

As shown, both the light source 182 and the optical receiver 184 are operably connected to the processor 160, which governs their operation. Also, the optical receiver 184 is operably coupled to provide the reflection data (from repository 192) to the memory 165 for storage and processing by reflection data classification logic 190. The reflection data classification logic 190 may be configured to: (i) identify which core fibers pertain to which of the received reflection data (from repository 192) and (ii) segregate the reflection data stored with a repository 192 provided from reflected light signals 150 pertaining to similar regions of the stylet 120 or spectral widths into analysis groups. The reflection data for each analysis group is made available to shape sensing logic 194 for analytics.

According to one embodiment of the disclosure, the shape sensing logic 194 is configured to compare wavelength shifts measured by sensors deployed in each periphery core fiber at the same measurement region of the stylet 120 (or same spectral width) to the wavelength shift at a center core fiber of the multi-core optical fiber 135 positioned along central axis and operating as a neutral axis of bending. From these analytics, the shape sensing logic 194 may determine the shape the core fibers have taken in 3D space and may further determine the current physical state of the catheter 121 in 3D space for rendering on the display 170.

According to one embodiment of the disclosure, the shape sensing logic 194 may generate a rendering of the current physical state of the stylet 120 (and potentially the catheter 121), based on heuristics or run-time analytics. For example, the shape sensing logic 194 may be configured in accordance with machine-learning techniques to access a data store (library) with pre-stored data (e.g., images, etc.) pertaining to different regions of the stylet 120 (or catheter 121) in which reflected light from core fibers have previously experienced similar or identical wavelength shifts. From the pre-stored data, the current physical state of the stylet 120 (or catheter 121) may be rendered. Alternatively, as another example, the shape sensing logic 194 may be configured to determine, during run-time, changes in the physical state of each region of the multi-core optical fiber 135 based on at least: (i) resultant wavelength shifts experienced by different core fibers within the optical fiber 135, and (ii) the relationship of these wavelength shifts generated by sensors positioned along different periphery core fibers at the same cross-sectional region of the multi-core optical fiber 135 to the wavelength shift generated by a sensor of the center core fiber at the same cross-sectional region. It is contemplated that other processes and procedures may be performed to utilize the wavelength shifts as measured by sensors along each of the core fibers within the multi-core optical fiber 135 to render appropriate changes in the physical state of the stylet 120 (and/or catheter 121), especially to enable guidance of the stylet 120, when positioned at a distal tip of the catheter 121, within the vasculature of the patient and at a desired destination within the body.

The console 110 may further include electrical signaling logic 181, which is positioned to receive one or more electrical signals from the stylet 120. The stylet 120 is configured to support both optical connectivity as well as electrical connectivity. The electrical signaling logic 181 receives the electrical signals (e.g., ECG signals) from the stylet 120 via the conductive medium. The electrical signals may be processed by electrical signal logic 196, executed by the processor 160, to determine ECG waveforms for display.

Referring to FIG. 1B, an alternative exemplary embodiment of a medical instrument monitoring system 100 is shown. Herein, the medical instrument monitoring system 100 features a console 110 and a medical instrument 130 communicatively coupled to the console 110. For this embodiment, the medical instrument 130 corresponds to a catheter, which features an integrated tubing with two or more lumen extending between a proximal end 131 and a distal end 132 of the integrated tubing. The integrated tubing (sometimes referred to as “catheter tubing”) is in communication with one or more extension legs 140 via a bifurcation hub 142. An optical-based catheter connector 144 may be included on a proximal end of at least one of the extension legs 140 to enable the catheter 130 to operably connect to the console 110 via an interconnect 145 or another suitable component. Herein, the interconnect 145 may include a connector 146 that, when coupled to the optical-based catheter connector 144, establishes optical connectivity between one or more optical fibers 147 (hereinafter, “optical fiber(s)”) included as part of the interconnect 145 and core fibers 137 deployed within the catheter 130 and integrated into the tubing. Alternatively, a different combination of connectors, including one or more adapters, may be used to optically connect the optical fiber(s) 147 to the core fibers 137 within the catheter 130. The core fibers 137 deployed within the catheter 130 as illustrated in FIG. 1B include the same characteristics and perform the same functionalities as the core fibers 137 deployed within the stylet 120 of FIG. 1A.

The optical logic 180 is configured to support graphical rendering of the catheter 130, most notably the integrated tubing of the catheter 130, based on characteristics of the reflected light signals 150 received from the catheter 130. The characteristics may include shifts in wavelength caused by strain on certain regions of the core fibers 137 integrated within (or along) a wall of the integrated tubing, which may be used to determine (through computation or extrapolation of the wavelength shifts) the physical state of the catheter 130, notably its integrated tubing or a portion of the integrated tubing such as a tip or distal end.

More specifically, the optical logic 180 includes a light source 182. The light source 182 is configured to transmit the broadband incident light 155 for propagation over the optical fiber(s) 147 included in the interconnect 145, which are optically connected to multiple core fibers 137 within the catheter tubing. Herein, the optical receiver 184 is configured to: (i) receive returned optical signals, namely reflected light signals 150 received from optical fiber-based reflective gratings (sensors) fabricated within each of the core fibers 137 deployed within the catheter 130, and (ii) translate the reflected light signals 150 into reflection data (from repository 192), namely data in the form of electrical signals representative of the reflected light signals including wavelength shifts caused by strain. The reflected light signals 150 associated with different spectral widths include reflected light signals 151 provided from sensors positioned in the center core fiber (reference) of the catheter 130 and reflected light signals 152 provided from sensors positioned in the outer core fibers of the catheter 130, as described below.

As noted above, the shape sensing logic 194 is configured to compare wavelength shifts measured by sensors deployed in each outer core fiber at the same measurement region of the catheter (or same spectral width) to the wavelength shift at the center core fiber positioned along central axis and operating as a neutral axis of bending. From these analytics, the shape sensing logic 190 may determine the shape the core fibers have taken in 3D space and may further determine the current physical state of the catheter 130 in 3D space for rendering on the display 170.

According to one embodiment of the disclosure, the shape sensing logic 194 may generate a rendering of the current physical state of the catheter 130, especially the integrated tubing, based on heuristics or run-time analytics. For example, the shape sensing logic 194 may be configured in accordance with machine-learning techniques to access a data store (library) with pre-stored data (e.g., images, etc.) pertaining to different regions of the catheter 130 in which the core fibers 137 experienced similar or identical wavelength shifts. From the pre-stored data, the current physical state of the catheter 130 may be rendered. Alternatively, as another example, the shape sensing logic 194 may be configured to determine, during run-time, changes in the physical state of each region of the catheter 130, notably the tubing, based on at least (i) resultant wavelength shifts experienced by the core fibers 137 and (ii) the relationship of these wavelength shifts generated by sensors positioned along different outer core fibers at the same cross-sectional region of the catheter 130 to the wavelength shift generated by a sensor of the center core fiber at the same cross-sectional region. It is contemplated that other processes and procedures may be performed to utilize the wavelength shifts as measured by sensors along each of the core fibers 137 to render appropriate changes in the physical state of the catheter 130.

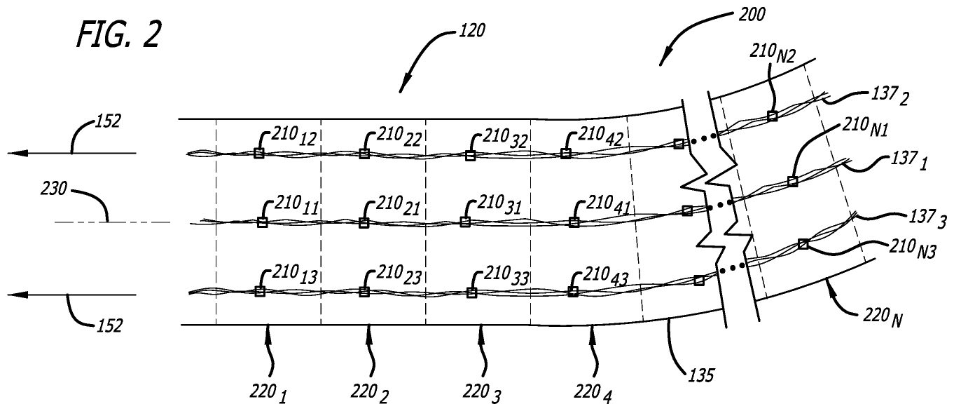

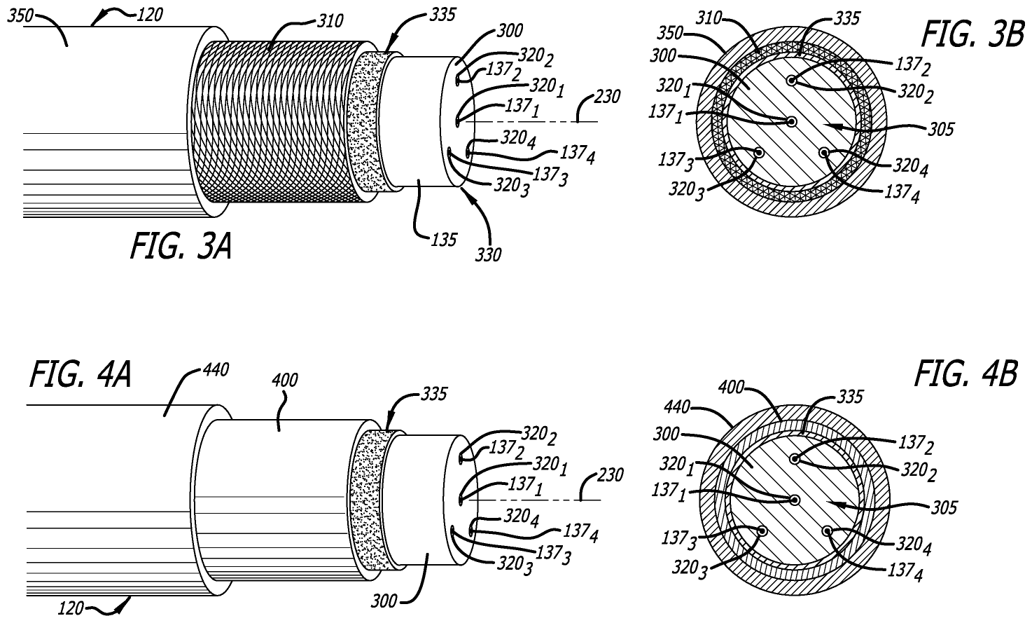

Referring to FIG. 2, an exemplary embodiment of a structure of a section of the multi-core optical fiber included within the stylet 120 of FIG. 1A is shown in accordance with some embodiments. The multi-core optical fiber section 200 of the multi-core optical fiber 135 depicts certain core fibers 137₁-137_{M} (M≥2, M=4 as shown, see FIG. 3A) along with the spatial relationship between sensors (e.g., reflective gratings) 210₁₁-210_{NM} (N≥2; M≥2) present within the core fibers 137₁-137_{M}, respectively. As noted above, the core fibers 137₁-137_{M} may be collectively referred to as “the core fibers 137.”

As shown, the section 200 is subdivided into a plurality of cross-sectional regions 220₁-220_{N}, where each cross-sectional region 220₁-220_{N} corresponds to reflective gratings 210₁₁-210₁₄ . . . 210_{N1}-210_{N4}. Some or all of the cross-sectional regions 220₁ . . . 220_{N} may be static (e.g., prescribed length) or may be dynamic (e.g., vary in size among the regions 220₁ . . . 220_{N}). A first core fiber 137₁ is positioned substantially along a center (neutral) axis 230 while core fiber 137₂ may be oriented within the cladding of the multi-core optical fiber 135, from a cross-sectional, front-facing perspective, to be position on “top” the first core fiber 137₁. In this deployment, the core fibers 137₃ and 137₄ may be positioned “bottom left” and “bottom right” of the first core fiber 137₁. As examples, FIGS. 3A-4B provides illustrations of such.

Referencing the first core fiber 137₁ as an illustrative example, when the stylet 120 is operative, each of the reflective gratings 210₁-210_{N} reflects light for a different spectral width. As shown, each of the gratings 210₁ᵢ-210_{Ni} (1≤i≤M) is associated with a different, specific spectral width, which would be represented by different center frequencies of f₁ . . . f_{N}, where neighboring spectral widths reflected by neighboring gratings are non-overlapping according to one embodiment of the disclosure.

Herein, positioned in different core fibers 137₂-137₃ but along at the same cross-sectional regions 220-220_{N} of the multi-core optical fiber 135, the gratings 210₁₂-210_{N2} and 210₁₃-210_{N3} are configured to reflect incoming light at same (or substantially similar) center frequency. As a result, the reflected light returns information that allows for a determination of the physical state of the optical fibers 137 (and the stylet 120) based on wavelength shifts measured from the returned, reflected light. In particular, strain (e.g., compression or tension) applied to the multi-core optical fiber 135 (e.g., at least core fibers 137₂-137₃) results in wavelength shifts associated with the returned, reflected light. Based on different locations, the core fibers 137₁-137₄ experience different types and degree of strain based on angular path changes as the stylet 120 advances in the patient.

For example, with respect to the multi-core optical fiber section 200 of FIG. 2, in response to angular (e.g., radial) movement of the stylet 120 is in the left-veering direction, the fourth core fiber 137₄ (see FIG. 3A) of the multi-core optical fiber 135 with the shortest radius during movement (e.g., core fiber closest to a direction of angular change) would exhibit compression (e.g., forces to shorten length). At the same time, the third core fiber 137₃ with the longest radius during movement (e.g., core fiber furthest from the direction of angular change) would exhibit tension (e.g., forces to increase length). As these forces are different and unequal, the reflected light from reflective gratings 210_{N2} and 210_{N3} associated with the core fiber 137₂ and 137₃ will exhibit different changes in wavelength. The differences in wavelength shift of the reflected light signals 150 can be used to extrapolate the physical configuration of the stylet 120 by determining the degrees of wavelength change caused by compression/tension for each of the periphery fibers (e.g., the second core fiber 137₂ and the third core fiber 137₃) in comparison to the wavelength of the reference core fiber (e.g., first core fiber 137₁) located along the neutral axis 230 of the multi-core optical fiber 135. These degrees of wavelength change may be used to extrapolate the physical state of the stylet 120. The reflected light signals 150 are reflected back to the console 110 via individual paths over a particular core fiber 137i-137_{M}.

Referring to FIG. 3A, a first exemplary embodiment of the stylet of FIG. 1A supporting both an optical and electrical signaling is shown in accordance with some embodiments. Herein, the stylet 120 features a centrally located multi-core optical fiber 135, which includes a cladding 300 and a plurality of core fibers 137₁-137_{M} (M≥2; M=4) residing within a corresponding plurality of lumens 320₁-320_{M}. While the multi-core optical fiber 135 is illustrated within four (4) core fibers 137₁-137₄, a greater number of core fibers 137₁-137_{M} (M>4) may be deployed to provide a more detailed 3D sensing of the physical state (e.g., shape, etc.) of the multi-core optical fiber 135 and the stylet 120 deploying the optical fiber 135.

For this embodiment of the disclosure, the multi-core optical fiber 135 is encapsulated within a concentric braided tubing 310 positioned over a low coefficient of friction layer 335. The braided tubing 310 may feature a “mesh” construction, in which the spacing between the intersecting conductive elements is selected based on the degree of rigidity desired for the stylet 120, as a greater spacing may provide a lesser rigidity, and thereby, a more pliable stylet 120.

According to this embodiment of the disclosure, as shown in FIGS. 3A-3B, the core fibers 137₁-137₄ include (i) a central core fiber 137₁ and (ii) a plurality of periphery core fibers 137₂-137₄, which are maintained within lumens 320₁-320₄ formed in the cladding 300. According to one embodiment of the disclosure, one or more of the lumen 320₁-320₄ may be configured with a diameter sized to be greater than the diameter of the core fibers 137₁-137₄. By avoiding a majority of the surface area of the core fibers 137₁-137₄ from being in direct physical contact with a wall surface of the lumens 320₁-320₄, the wavelength changes to the incident light are caused by angular deviations in the multi-core optical fiber 135 thereby reducing influence of compression and tension forces being applied to the walls of the lumens 320₁-320_{M}, not the core fibers 137₁-137_{M} themselves.

As further shown in FIGS. 3A-3B, the core fibers 137₁-137₄ may include central core fiber 137₁ residing within a first lumen 320₁ formed along the first neutral axis 230 and a plurality of core fibers 137₂-137₄ residing within lumens 320₂-320₄ each formed within different areas of the cladding 300 radiating from the first neutral axis 230. In general, the core fibers 137₂-137₄, exclusive of the central core fiber 137₁, may be positioned at different areas within a cross-sectional area 305 of the cladding 300 to provide sufficient separation to enable 3D sensing of the multi-core optical fiber 135 based on changes in wavelength of incident light propagating through the core fibers 137₂-137₄ and reflected back to the console for analysis.

For example, where the cladding 300 features a circular cross-sectional area 305 as shown in FIG. 3B, the core fibers 137₂-137₄ may be positioned substantially equidistant from each other as measured along a perimeter of the cladding 300, such as at “top” (12 o'clock), “bottom-left” (8 o'clock) and “bottom-right” (4 o'clock) locations as shown. Hence, in general terms, the core fibers 137₂-137₄ may be positioned within different segments of the cross-sectional area 305. Where the cross-sectional area 305 of the cladding 300 has a distal tip 330 and features a polygon cross-sectional shape (e.g., triangular, square, rectangular, pentagon, hexagon, octagon, etc.), the central core fiber 137₁ may be located at or near a center of the polygon shape, while the remaining core fibers 137₂-137_{M} may be located proximate to angles between intersecting sides of the polygon shape.

Referring still to FIGS. 3A-3B, operating as the conductive medium for the stylet 120, the braided tubing 310 provides mechanical integrity to the multi-core optical fiber 135 and operates as a conductive pathway for electrical signals. For example, the braided tubing 310 may be exposed to a distal tip of the stylet 120. The cladding 300 and the braided tubing 310, which is positioned concentrically surrounding a circumference of the cladding 300, are contained within the same insulating layer 350. The insulating layer 350 may be a sheath or conduit made of protective, insulating (e.g., non-conductive) material that encapsulates both for the cladding 300 and the braided tubing 310, as shown.

Referring to FIG. 4A, a second exemplary embodiment of the stylet of FIG. 1B is shown in accordance with some embodiments. Referring now to FIG. 4A, a second exemplary embodiment of the stylet 120 of FIG. 1B supporting both an optical and electrical signaling is shown. Herein, the stylet 120 features the multi-core optical fiber 135 described above and shown in FIG. 3A, which includes the cladding 300 and the first plurality of core fibers 137₁-137_{M} (M≥3; M=4 for embodiment) residing within the corresponding plurality of lumens 320i-320_{M}. For this embodiment of the disclosure, the multi-core optical fiber 135 includes the central core fiber 137₁ residing within the first lumen 320₁ formed along the first neutral axis 230 and the second plurality of core fibers 137₂-137₄ residing within corresponding lumens 320₂-320₄ positioned in different segments within the cross-sectional area 305 of the cladding 300. Herein, the multi-core optical fiber 135 is encapsulated within a conductive tubing 400. The conductive tubing 400 may feature a “hollow” conductive cylindrical member concentrically encapsulating the multi-core optical fiber 135.

Referring to FIGS. 4A-4B, operating as a conductive medium for the stylet 120 in the transfer of electrical signals (e.g., ECG signals) to the console, the conductive tubing 400 may be exposed up to a tip 410 of the stylet 120. For this embodiment of the disclosure, a conductive epoxy 420 (e.g., metal-based epoxy such as a silver epoxy) may be affixed to the tip 410 and similarly joined with a termination/connection point created at a proximal end 430 of the stylet 120. The cladding 300 and the conductive tubing 400, which is positioned concentrically surrounding a circumference of the cladding 300, are contained within the same insulating layer 440. The insulating layer 440 may be a protective conduit encapsulating both for the cladding 300 and the conductive tubing 400, as shown.

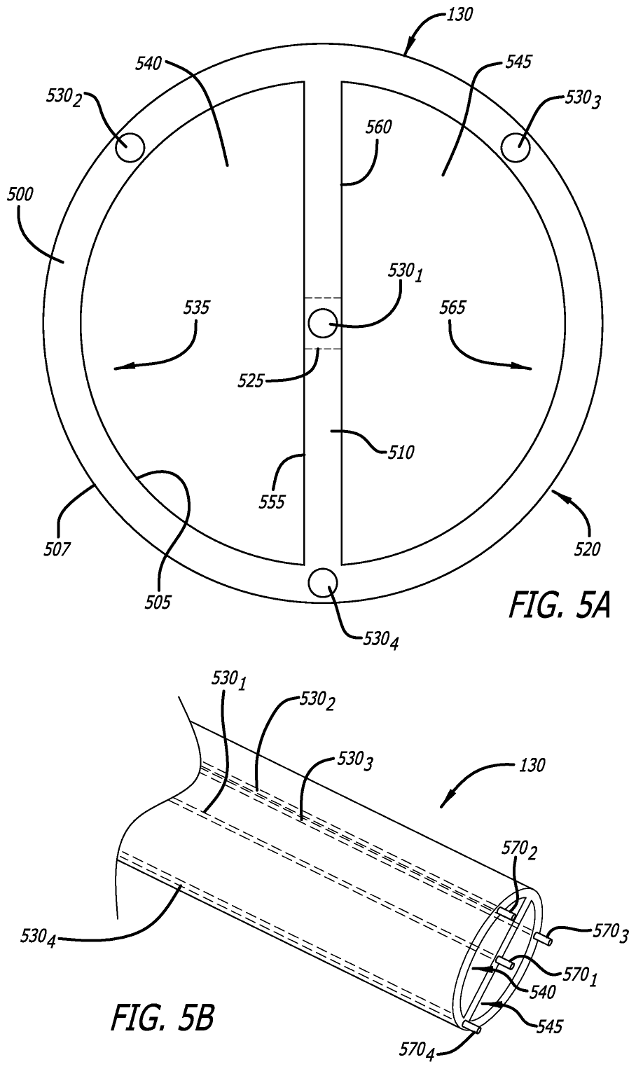

Referring to FIG. 5A, an elevation view of a first illustrative embodiment of a catheter including integrated tubing, a diametrically disposed septum, and micro-lumens formed within the tubing and septum is shown in accordance with some embodiments. Herein, the catheter 130 includes integrated tubing, the diametrically disposed septum 510, and the plurality of micro-lumens 530₁-530₄ which, for this embodiment, are fabricated to reside within the wall 500 of the integrated tubing of the catheter 130 and within the septum 510. In particular, the septum 510 separates a single lumen, formed by the inner surface 505 of the wall 500 of the catheter 130, into multiple lumen, namely two lumens 540 and 545 as shown. Herein, the first lumen 540 is formed between a first arc-shaped portion 535 of the inner surface 505 of the wall 500 forming the catheter 130 and a first outer surface 555 of the septum 510 extending longitudinally within the catheter 130. The second lumen 545 is formed between a second arc-shaped portion 565 of the inner surface 505 of the wall 500 forming the catheter 130 and a second outer surfaces 560 of the septum 510.

According to one embodiment of the disclosure, the two lumens 540 and 545 have approximately the same volume. However, the septum 510 need not separate the tubing into two equal lumens. For example, instead of the septum 510 extending vertically (12 o'clock to 6 o'clock) from a front-facing, cross-sectional perspective of the tubing, the septum 510 could extend horizontally (3 o'clock to 9 o'clock), diagonally (1 o'clock to 7 o'clock; 10 o'clock to 4 o'clock) or angularly (2 o'clock to 10 o'clock). In the later configuration, each of the lumens 540 and 545 of the catheter 130 would have a different volume.

With respect to the plurality of micro-lumens 530₁-530₄, the first micro-lumen 530₁ is fabricated within the septum 510 at or near the cross-sectional center 525 of the integrated tubing. For this embodiment, three micro-lumens 530₂-530₄ are fabricated to reside within the wall 500 of the catheter 130. In particular, a second micro-lumen 530₂ is fabricated within the wall 500 of the catheter 130, namely between the inner surface 505 and outer surface 507 of the first arc-shaped portion 535 of the wall 500. Similarly, the third micro-lumen 530₃ is also fabricated within the wall 500 of the catheter 130, namely between the inner and outer surfaces 505/507 of the second arc-shaped portion 555 of the wall 500. The fourth micro-lumen 530₄ is also fabricated within the inner and outer surfaces 505/507 of the wall 500 that are aligned with the septum 510.

According to one embodiment of the disclosure, as shown in FIG. 5A, the micro-lumens 530₂-530₄ are positioned in accordance with a “top-left” (10 o'clock), “top-right” (2 o'clock) and “bottom” (6 o'clock) layout from a front-facing, cross-sectional perspective. Of course, the micro-lumens 530₂-530₄ may be positioned differently, provided that the micro-lumens 530₂-530₄ are spatially separated along the circumference 520 of the catheter 130 to ensure a more robust collection of reflected light signals from the outer core fibers 570₂-570₄ when installed. For example, two or more of micro-lumens (e.g., micro-lumens 530₂ and 530₄) may be positioned at different quadrants along the circumference 520 of the catheter wall 500.

Referring to FIG. 5B, a perspective view of the first illustrative embodiment of the catheter of FIG. 5A including core fibers installed within the micro-lumens is shown in accordance with some embodiments. According to one embodiment of the disclosure, the second plurality of micro-lumens 530₂-530₄ are sized to retain corresponding outer core fibers 570₂-570₄, where the diameter of each of the second plurality of micro-lumens 530₂-530₄ may be sized just larger than the diameters of the outer core fibers 570₂-570₄. The size differences between a diameter of a single core fiber and a diameter of any of the micro-lumen 530₁-530₄ may range between 0.001 micrometers (μm) and 1000 μm, for example. As a result, the cross-sectional areas of the outer core fibers 570₂-570₄ would be less than the cross-sectional areas of the corresponding micro-lumens 530₂-530₄. A “larger” micro-lumen (e.g., micro-lumen 530₂) may better isolate external strain being applied to the outer core fiber 570₂ from strain directly applied to the catheter 130 itself. Similarly, the first micro-lumen 530₁ may be sized to retain the center core fiber 570₁, where the diameter of the first micro-lumen 530₁ may be sized just larger than the diameter of the center core fiber 570₁.

As an alternative embodiment of the disclosure, one or more of the micro-lumens 530₁-530₄ may be sized with a diameter that exceeds the diameter of the corresponding one or more core fibers 570₁-570₄. However, at least one of the micro-lumens 530₁-530₄ is sized to fixedly retain their corresponding core fiber (e.g., core fiber retained with no spacing between its lateral surface and the interior wall surface of its corresponding micro-lumen). As yet another alternative embodiment of the disclosure, all the micro-lumens 530₁-530₄ are sized with a diameter to fixedly retain the core fibers 570₁-570₄.

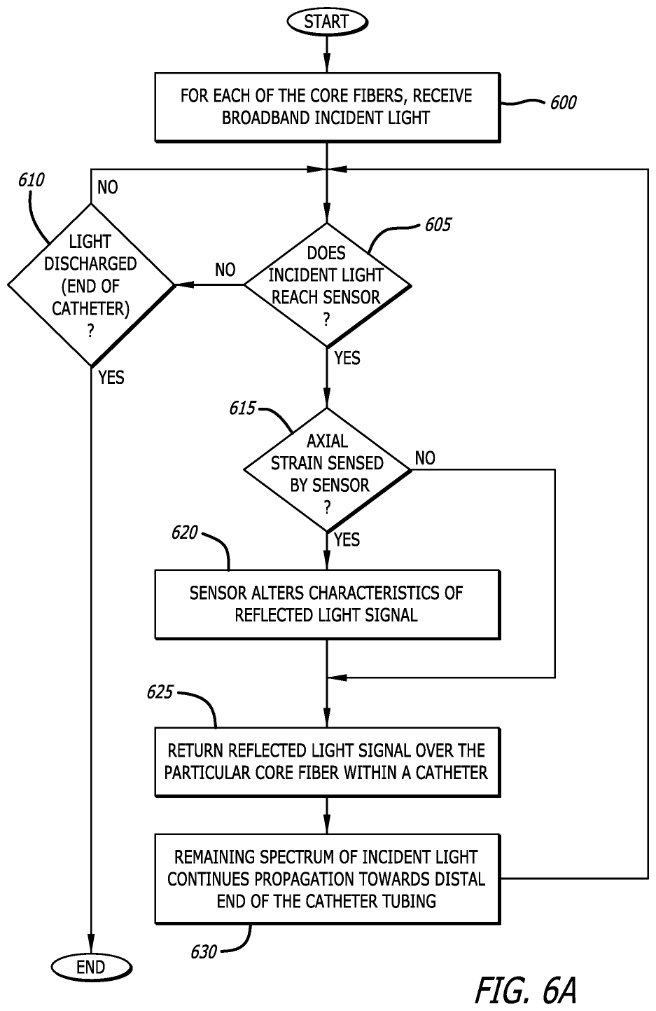

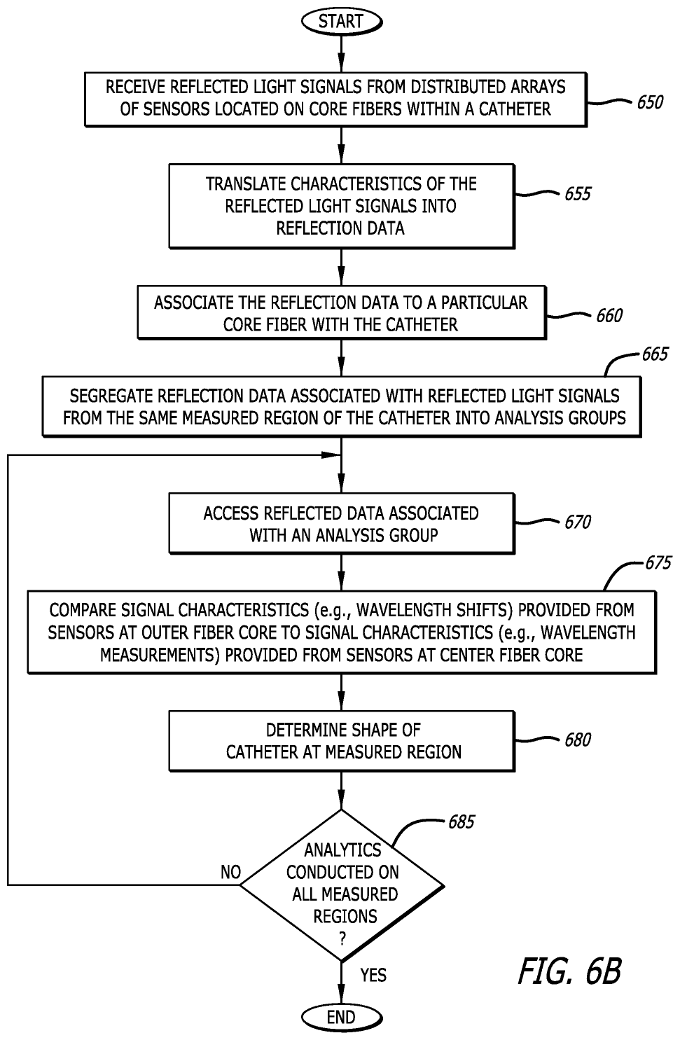

Referring to FIGS. 6A-6B, flowcharts of methods of operations conducted by the medical instrument monitoring system of FIGS. 1A-1B to achieve optic 3D shape sensing are shown in accordance with some embodiments. Herein, the catheter includes at least one septum spanning across a diameter of the tubing wall and continuing longitudinally to subdivide the tubing wall. The medial portion of the septum is fabricated with a first micro-lumen, where the first micro-lumen is coaxial with the central axis of the catheter tubing. The first micro-lumen is configured to retain a center core fiber. Two or more micro-lumen, other than the first micro-lumen, are positioned at different locations circumferentially spaced along the wall of the catheter tubing. For example, two or more of the second plurality of micro-lumens may be positioned at different quadrants along the circumference of the catheter wall.

Furthermore, each core fiber includes a plurality of sensors spatially distributed along its length between at least the proximal and distal ends of the catheter tubing. This array of sensors is distributed to position sensors at different regions of the core fiber to enable distributed measurements of strain throughout the entire length or a selected portion of the catheter tubing. These distributed measurements may be conveyed through reflected light of different spectral widths (e.g., specific wavelength or specific wavelength ranges) that undergoes certain wavelength shifts based on the type and degree of strain.

According to one embodiment of the disclosure, as shown in FIG. 6A, for each core fiber, broadband incident light is supplied to propagate through a particular core fiber (block 600). Unless discharged, upon the incident light reaching a sensor of a distributed array of sensors measuring strain on a particular core fiber, light of a prescribed spectral width associated with the first sensor is to be reflected back to an optical receiver within a console (blocks 605-610). Herein, the sensor alters characteristics of the reflected light signal to identify the type and degree of strain on the particular core fiber as measured by the first sensor (blocks 615-620). According to one embodiment of the disclosure, the alteration in characteristics of the reflected light signal may signify a change (shift) in the wavelength of the reflected light signal from the wavelength of the incident light signal associated with the prescribed spectral width. The sensor returns the reflected light signal over the core fiber and the remaining spectrum of the incident light continues propagation through the core fiber toward a distal end of the catheter tubing (blocks 625-630). The remaining spectrum of the incident light may encounter other sensors of the distributed array of sensors, where each of these sensors would operate as set forth in blocks 605-630 until the last sensor of the distributed array of sensors returns the reflected light signal associated with its assigned spectral width and the remaining spectrum is discharged as illumination.

Referring now to FIG. 6B, during operation, multiple reflected light signals are returned to the console from each of the plurality of core fibers residing within the corresponding plurality of micro-lumens formed within a catheter, such as the catheter of FIG. 1B. In particular, the optical receiver receives reflected light signals from the distributed arrays of sensors located on the center core fiber and the outer core fibers and translates the reflected light signals into reflection data, namely electrical signals representative of the reflected light signals including wavelength shifts caused by strain (blocks 650-655). The reflection data classification logic is configured to identify which core fibers pertain to which reflection data and segregate reflection data provided from reflected light signals pertaining to a particular measurement region (or similar spectral width) into analysis groups (block 660-665).

Each analysis group of reflection data is provided to shape sensing logic for analytics (block 670). Herein, the shape sensing logic compares wavelength shifts at each outer core fiber with the wavelength shift at the center core fiber positioned along central axis and operating as a neutral axis of bending (block 675). From this analytics, on all analytic groups (e.g., reflected light signals from sensors in all or most of the core fibers), the shape sensing logic may determine the shape the core fibers have taken in 3D space, from which the shape sensing logic can determine the current physical state of the catheter in three-dimension space (blocks 680-685).

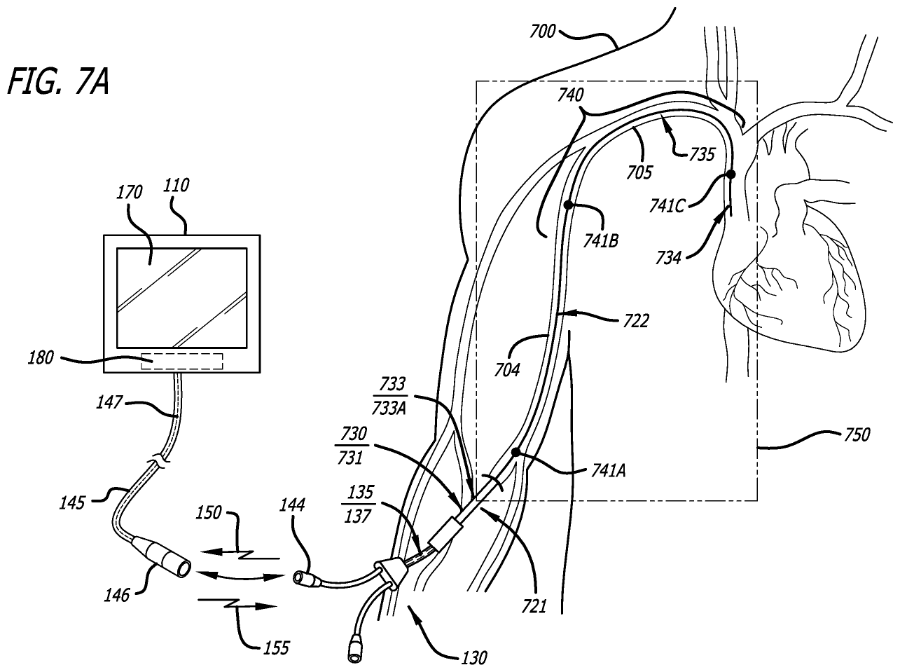

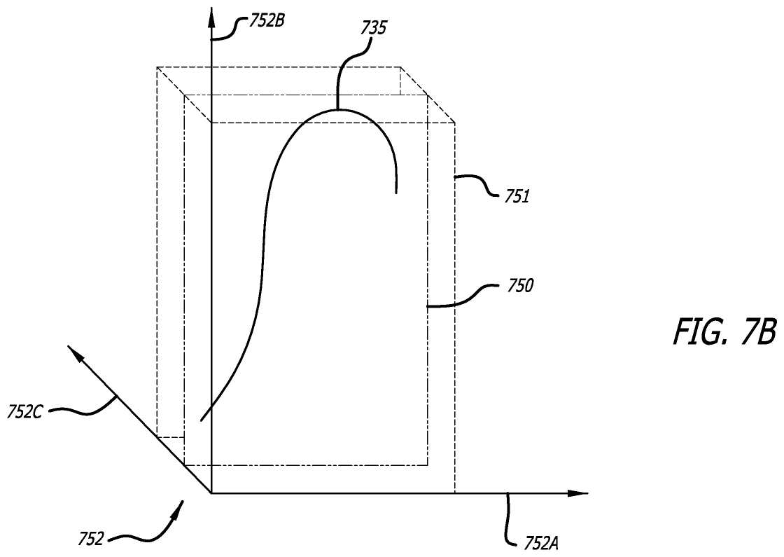

Referring to FIG. 7A, an exemplary embodiment of the medical instrument monitoring system of FIG. 1B during operation and insertion of the catheter into a patient are shown in accordance with some embodiments. Herein, the catheter 130 generally includes the integrated tubing of the catheter 130 with a proximal portion 721 that generally remains exterior to the patient 700 and a distal portion 722 that generally resides within the patient vasculature after placement is complete. The (integrated) catheter tubing of the catheter 130 may be advanced to a desired position within the patient vasculature such that a distal end (or tip) 734 of the catheter tubing of the catheter 130 is proximate the patient's heart, such as in the lower one-third (⅓) portion of the Superior Vena Cava (“SVC”) for example. In some embodiments, various instruments may be disposed at the distal end 734 of the catheter 130 to measure pressure of blood in a certain heart chamber and in the blood vessels, view an interior of blood vessels, or the like. In alternative embodiments, such as those that utilize the stylet assembly of FIG. 1A and the catheter 121, such instruments may be disposed at a distal end of the stylet 120.

During advancement through a patient vasculature, the catheter tubing of the catheter 130 receives broadband incident light 155 from the console 110 via optical fiber(s) 147 within the interconnect 145, where the incident light 155 propagates along the core fibers 137 of the multi-core optical fiber 135 within the catheter tubing of the catheter 130. According to one embodiment of the disclosure, the connector 146 of the interconnect 145 terminating the optical fiber(s) 147 may be coupled to the optical-based catheter connector 144, which may be configured to terminate the core fibers 137 deployed within the catheter 130. Such coupling optically connects the core fibers 137 of the catheter 130 with the optical fiber(s) 147 within the interconnect 145. The optical connectivity is needed to propagate the incident light 155 to the core fibers 137 and return the reflected light signals 150 to the optical logic 180 within the console 110 over the interconnect 145. As described below in detail, the physical state of the catheter 130 may be ascertained based on analytics of the wavelength shifts of the reflected light signals 150 where the physical state includes a 3D shape 735 of the optical fiber 135.

In some embodiments, the system 100 may include a guide 730 having a straight section 731. The guide 730 may be formed of an introducer having a lumen through which the catheter 130 is inserted. In use a distal portion of the guide 730 may be disposed inside the patient 700 while a proximal portion remains outside the patient 700.

In some implementations, the guide 730, or more specifically the straight section 731, may facilitate a calibration of the optical fiber 135. For example, while a section 733 of the optical fiber 135 is disposed within the straight section 731, the shape framing logic 195 may interpret shape data pertaining to the shape 733A of the section 733 as defining a straight line.

The shape framing logic 195 may define a reference plane 750 in accordance with one or more portions of the 3D shape 735, and the reference plane 750 may define a reference frame 751 for displaying an image of the 3D shape 735. For example, according to one implementation, a curved segment 740 of the optical fiber 135/catheter 130 may define the reference plane 750. In other words, the shape framing logic 195 may process the shape of the curved segment 740 to define a plane estimated by the curved segment 740. By way of example, the catheter 130 may be a peripherally inserted central catheter (PICC) to be positioned along the curved transition between the basilic vein 704 and the subclavian vein 705 when the PICC inserted. As such, the shape framing logic 195 may identify the curved transition between the basilic vein 704 and the subclavian vein 705 as the curved segment 740.

According to an alternative implementation, the shape framing logic 195 may define the reference plane 750 in accordance with three or more points (e.g., 741A, 741B, and 741C) disposed along the 3D shape 735. In some embodiments, the three points 741A, 741B, 741C may be predefined along the 3D shape 735, such as proximal end point, a center point and a distal end point, for example. In other embodiments, the clinician may select the three points on an image of the 3D shape 735, via an input device (e.g., a computer mouse). In still other embodiments, the shape framing logic 195 may automatically identify the three points in relation to the curved segment 740. In some embodiments, the reference plane 750 may be defined such that the three points 741A, 741B, 741C are equidistant from the reference plane 750. As may be appreciated by one of ordinary skill, other geometric techniques may be utilized to define the plane 750.

With further reference to FIG. 7A the catheter 130 is illustrated in accordance with a front view of the patient 700. As such, the 3D shape 735 as illustrated may be a front view of the 3D shape 735 consistent with the front view of the patient 700. In some instances, the reference plane 750 may be substantially parallel with the front side of the patient 700. Therefore, a front view of the 3D shape 735 may be substantially consistent with viewing the 3D shape 735 at angle perpendicular to the reference plane 750.

FIG. 7B illustrates the 3D shape 735 as may oriented with respect to a reference frame, in accordance with some embodiments. The shape framing logic 195 may define a reference frame 751 for viewing an image of the 3D shape 735 on the display 170. For example, the reference frame 751 may define various views of the 3D shape 735 (e.g., a front view, a top view, a right side, etc.) For illustrative purposes, the reference frame 751 is shown in accordance with a 3D coordinate axis system 752 having a horizontal x-axis 752A pointing to the right, a vertical y-axis 752B pointing up, and a z-axis 752C pointing into the page. The 3D shape 735 is shown together with the reference plane 750. In the illustrated embodiment, the reference plane 750 is oriented in relation to the reference frame 751 so that the reference plane 750 is in parallel with the x-y plane. As such, a front view of the 3D shape 735 is defined by a viewing angle in the direction of the z-axis 752C, a bottom view of the 3D shape 735 is defined by a viewing angle in the direction of the y-axis 752B, and left side view is defined by a viewing angle in the direction of the x-axis 752A. Similarly, back view of the 3D shape 735 is defined by a viewing angle in the opposite direction of the z-axis 752C, a top view of the 3D shape 735 is defined by a viewing angle in the opposite direction of the y-axis 752B, and right side view is defined by a viewing angle in the opposite direction of the x-axis 752A.

The shape framing logic 195 is configured to render an image of the 3D shape 735 at any of the viewing angles described above. The shape framing logic 195 may also facilitate rendering an image of the 3D shape 735 at any angle with respect to the reference frame 751 as may be defined by the operator via the input device. In other words, the clinician may manipulate the orientation of the 3D shape 735 to view an image 3D shape 735 from any angle. In some embodiments, the shape framing logic 195 may fix the orientation of the 3D shape 735 and/or the reference plane 750 with respect to the reference frame 751.

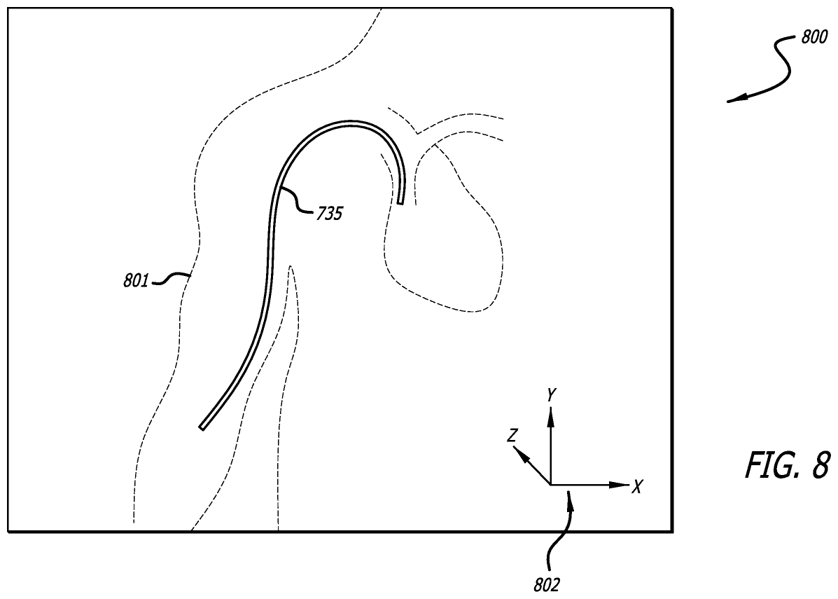

FIG. 8 illustrates an exemplary screen shot 800 showing an image of the 3D shape 735 of FIGS. 7A, 7B rendered according to the reference frame 751. In some embodiments, the screen shot 800 may include a representation 801 of a patient body. For example, in the illustrated embodiment, the representation 801 includes an outline of a typical patient body as may be viewed from the front to indicate the orientation of the 3D shape 735. In some embodiments, the representation 801 may include representations of other body parts such as the heart as illustrated. In still other embodiments, the screen shot 800 may include indicia 801 to indicate the orientation of the 3D shape 735 as defined by the reference frame 751, such a coordinate axis system, for example. Images of the 3D shape 735 are not limited to the front view as illustrated in FIG. 8. Although not shown, the shape framing logic 195 may facilitate rendering of images of the 3D shape 735 at any orientation via input from the clinician. In such embodiments, the representation 801 and/or the indica 802 may provide indication to the clinician as to the rendered orientation of the 3D shape 735.

In some embodiments, the system 100 may be communicatively coupled with an imaging system (e.g., ultrasound, MRI, X-ray, etc., not shown), and the shape framing logic 195 may facilitate rendering the image of the 3D shape 735 along with an image of patient. In some instances, the clinician may orient and/or position the image of the 3D shape 735 to position a portion of the 3D shape 735, such as a catheter tip, for example, at a specific location relative to an image of the patient. As the imaging system may include an image of the medical device directly, such an image may facilitate visual comparison between the 3D shape 735 and the image of the medical device.

In further embodiments, other device location or tracking modalities may be coupled with the system 100 and employed to indicate a position of the catheter 130. Such modalities may include ECG signal monitoring as described above and magnetic field sensing such as described in U.S. Pat. No. 5,099,845 entitled “Medical Instrument Location Means,” which is incorporated herein by reference in its entirety. As such, the system 100 may render images or information on the display 170 pertaining to device location or tracking data in combination with the image of the 3D shape 735.

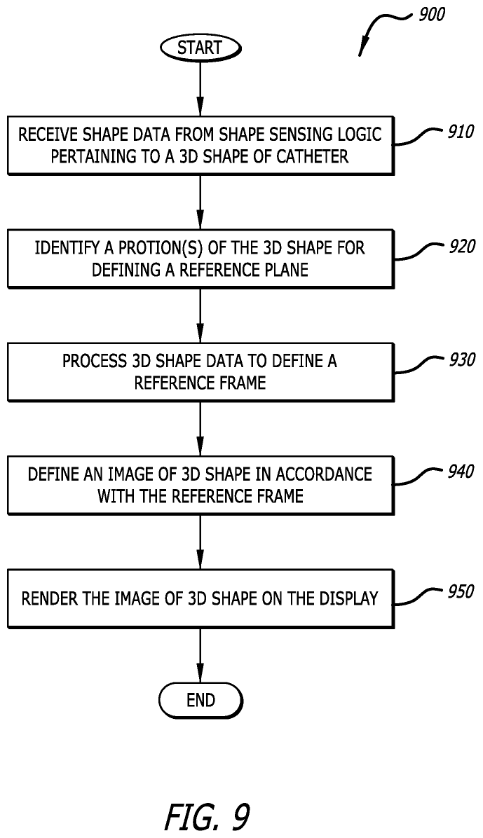

Referring to FIG. 9, a flowchart of a method of operations conducted by the medical instrument monitoring system of FIGS. 1A-1B to render an image of the 3D shape 735 on the display is shown, in accordance with some embodiments. The method 900 may be performed by the shape framing logic 195. In other embodiments, the shape framing logic 195 may be incorporated into the shape sensing logic 194, and as such, the method 900 may be performed by the shape sensing logic 194. The method 900 generally processes shape data to render an image of the 3D shape on the display. According to one embodiment of the disclosure, as shown in FIG. 9, the shape framing logic 195 receives 3D shape data pertaining to the 3D shape of the catheter from the shape sensing logic 194 (block 910).

The shape framing logic 195 then identifies a portion(s) of the 3D shape for defining a reference plane (block 920). The identification may include comparing portions of the 3D shape with one or more predefined shapes in memory. The predefined shapes in memory may correlate with shapes of anatomical elements within the patient body at their understood locations. In some implementations, the predefined shapes in memory may be established in accordance with boundary conditions based on typical anatomy across a population.

For example, the predefined shape in memory may include a curved portion, and the shape framing logic 195 search the 3D shape to identify a portion of the 3D shape that is consistent with the curved shape in memory. As a result of identifying a portion of the 3D shape that is consistent with the curved shape in memory, the shape framing logic 915 may choose three or more points along the identified portion of the 3D shape to define the reference plane. According to one embodiment, the predefined shape in memory may be consistent with a curve of the 3D shape defined by the vasculature of the patient, such as the vasculature extending between a basilic vein and a subclavian vein, for example.

In some embodiments, the system may include a plurality of curved shapes in memory that pertain to a plurality of medical procedures. In such embodiments, the shape framing logic 195 may receive input from the clinician pertaining to the medical procedure, including an insertion site for the medical device. Input from the clinician may include (i) a location of the insert site of the medical device including a right vs left side of the patient, (ii) a type of medical device (e.g., central venous catheter, infusion port, or PICC), (iii) an orientation of the patient, or (iv) an orientation of body part (e.g., arm). The shape framing logic 195 may select a curved shape from the plurality of curved shapes in memory in accordance with the input from the clinician, and compare the curved portion of the 3D shape with the selected curved shape.

The shape framing logic 195 may then define a reference frame for the 3D image from the identified portion of the 3D shape (block 930). According to one embodiment, the shape framing logic 195 may initially define a plane in accordance with the identified portion described above in relation to (block 920). As may be appreciated by one of ordinary skill, the plane may be defined according to various geometric techniques, e.g., three points, a line and a point, or a line and a direction. For example, the shape framing logic 195 may define the reference plane in accordance with three points disposed along an identified portion of the 3D shape and then define the reference frame indicating an orientation of the 3D shape (e.g., front to back, top to bottom, left side to right side) of the 3D shape. In some instances, the reference frame may be defined such that a front side of the reference frame is in parallel with the plane.

The shape framing logic 195 may then define an image of the 3D shape in accordance with the reference frame (block 940). In other words, the shape framing logic 195 may define an image of the 3D shape that may viewed on the display from one or more viewpoints with respect to the reference frame, i.e., from the front, top, right side, etc. In some embodiments, the shape framing logic 195 may define an image of the 3D shape viewable from any direction with respect the reference frame. The shape framing logic 195 may then render the image of the 3D shape on the display (block 950).

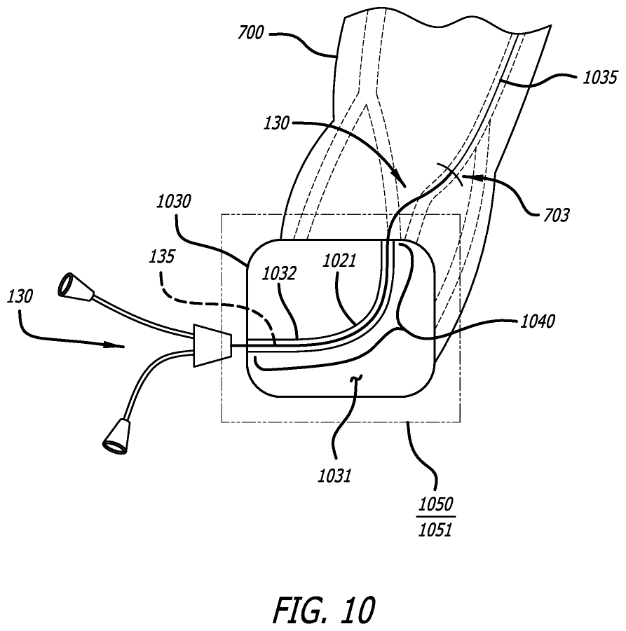

FIG. 10 illustrates an embodiment of a reference guide 1030 for defining a reference plane for a 3D shape 1035 which may be defined by the shape sensing logic 194 in a manner similar to the 3D shape 735 of FIGS. 7A and 7B. The reference guide 1030 is configured to define the plane 1050 and the resulting reference frame 1051. In the illustrated embodiment, the reference guide 1030 includes a plate 1031 defining a plane. The plate 1031 includes a groove 1032 disposed along a top surface of the plate 1031 defining a guideway, and the groove 1032 is configured to receive a segment of the catheter 130. A proximal portion 1021 of the catheter 130, disposed outside of the patient 700, is placed within the groove 1032 to define a curved segment 1040 of the catheter 130 (i.e., the optical fiber 135).

Similar to the curved segment 740 of FIG. 7A, the curved segment 1040 may define the plane 1050. In other words, the shape framing logic 195 may process shape data of the curved segment 1040 to define the plane 1050 as geometrically estimated by the curved segment 1040. With the reference plane 1050 defined, the shape framing logic 195 may then define the reference frame 1051, which may in some respects resemble the reference frame 751 of FIGS. 7A and 7B, for viewing an image of the 3D shape 1035 on the display 170. In the illustrated embodiment, as the curved segment 1040 is disposed within the groove 1032, the plane 1050 is in parallel with the plate 1031. The form of the reference guide 1040 is not limited to a flat plate, i.e., the reference guide 1030 may take any form suitable for defining the curved shape 1040.

In use, the clinician inserts the catheter 130 within the patient 700. The clinician places the proximal portion 1021 of the catheter 130 with the groove 1032 to define the curved segment 1040. The shape sensing logic 194 determines a 3D shape 1035 of the catheter 130, and the shape framing logic 195 determines the plane 1050 in accordance with the curved segment 1040 (i.e., the portion of the 3D shape 1035 extending along the curved segment 1040). The shape framing logic 195 defines the reference frame 1051 in accordance with the plane 1050 and renders an image of the 3D shape 1035 on the display 170.

The clinician may orient the reference frame 1051 via orientation of the reference guide 1030. As the reference guide 1030 and, by association, the curved segment 1040 are disposed outside the patient 700, the clinician may orient the reference guide 1030 to define a viewpoint of the 3D shape 1035. For example, the clinician may orient the reference guide 1030 to be in parallel with a front side of the patient 700 to define front view of the 3D shape 1035. In short, the clinician may orient the reference guide 1030 to facilitate viewing of the image of the 3D shape 1035 on the display 170 from any angle by adjusting the orientation of the reference guide 1030.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A medical device system comprising:
a medical device comprising an optical fiber having one or more of core fibers, each of the one or more core fibers including a plurality of sensors distributed along a longitudinal length of a corresponding core fiber, each sensor of the plurality of sensors configured to:
(i) reflect a light signal of a different spectral width based on received incident light, and
(ii) change a characteristic of the reflected light signal based on strain experienced by the optical fiber; and
a console including one or more processors and a non-transitory computer-readable medium having stored thereon logic, when executed by the one or more processors, causes operations including:
providing an incident light signal to the optical fiber;
receiving reflected light signals of different spectral widths of the incident light by one or more of the plurality of sensors;
processing the reflected light signals associated with the one or more of core fibers to determine a three-dimensional (3D) shape of the optical fiber;
defining a reference frame for displaying an image of the 3D shape;
orienting the 3D shape within the reference frame; and
rendering an image of the 3D shape on a display of the system in accordance with the reference frame.

2. The system according to claim 1, wherein orienting the 3D shape includes:
defining the reference plane according to a portion of the 3D shape;
fixing the 3D shape to the reference plane; and
orienting the reference plane with respect to the reference frame.

3. The system according to claim 2, wherein:
the portion of the 3D shape includes three or more points disposed along the 3D shape, and
the three or more points are equidistant from the reference plane.

4. The system according to claim 1, further comprising:
a guide including a lumen extending along a straight section of the guide, wherein:
in use, the optical fiber is inserted within the lumen, and
the operations further include calibrating the optical fiber in accordance with the straight section.

5. The system according to claim 2, wherein the operations further include orienting the reference plane with respect to the reference frame so that a front view image of the 3D shape according to the reference plane is aligned with a front view according to the reference frame.

6. The system according to claim 2, wherein the operations further include fixing the reference plane with respect to the reference frame.

7. The system according to claim 2, wherein the operations further include:
comparing a curved portion of the 3D shape with a curved shape stored in memory; and
as a result of the comparison, identifying the three or more points from the curved portion to define the reference plane when the curved portion of the 3D shape is consistent with the curved shape stored in memory.

8. The system according to claim 7, wherein in use, the curved portion of the 3D shape is disposed along a basilic vein, a subclavian vein, an innominate vein, or a superior vena cava of the patient.

9. The system according to claim 7, further comprising:
a plurality of curved shapes stored in memory, the plurality of curved shapes pertaining to a plurality of different insertion sites for the medical device, wherein the operations further include:
receiving input from a clinician defining an insertion site for the medical device;
selecting the curved shape from the plurality of curved shapes, the selected curved shape pertaining to the defined insertion site; and
comparing the curved portion of the 3D shape with the selected curved shape.

10. The system according to claim 9, wherein the input further defines the insertion site as located on a right side or a left side of the patient.

11. The system according to claim 7, further comprising a reference guide coupled with the medical device, wherein the curved portion of the 3D shape is disposed along a pathway of the reference guide.

12. The system according to claim 11, wherein an orientation of the reference guide with respect to the patient defines an orientation of the 3D shape with respect to the patient.

13. The system according to claim 12, wherein in use, the reference guide is displaced between a first guide orientation and a second guide orientation with respect to the patient to move the 3D shape between a first 3D shape orientation and a second 3D shape orientation with respect to the patient.

14. The system according to claim 1, wherein:
the system is coupled with a patient imaging system, and
the operations further include:
receiving image data from the patient imaging system; and
rendering an image of the patient on the display along with an image of the 3D shape.

15. The system according to claim 1, wherein the medical device is one of an introducer wire, a guidewire, a stylet, a stylet within a needle, a needle with the optical fiber inlayed into a cannula of the needle or a catheter with the optical fiber inlayed into one or more walls of the catheter.

16. A method for detecting placement of a medical device within a patient body, the method comprising:
providing an incident light signal to an optical fiber included within the medical device, wherein the optical fiber includes a one or more of core fibers, each of the one or more of core fibers including a plurality of reflective gratings distributed along a longitudinal length of a corresponding core fiber and each of the plurality of reflective gratings being configured to (i) reflect a light signal of a different spectral width based on received incident light, and (ii) change a characteristic of the reflected light signal based on strain experienced by the optical fiber;
receiving reflected light signals of different spectral widths of the incident light by one or more of the plurality of sensors;
processing the reflected light signals associated with the one or more of core fibers to determine a three-dimensional (3D) shape of the optical fiber;
defining a reference frame for displaying an image of the 3D shape;
orienting the 3D shape within the reference frame; and
rendering an image of the 3D shape on a display of a system in accordance with the reference frame.

17. The method according to claim 16, wherein orienting the 3D shape includes:
defining a reference plane according to a portion of the 3D shape;
fixing the 3D shape to the reference plane; and
orienting the reference plane with respect to the reference frame.

18. The method according to claim 17, wherein:
the portion of the 3D shape includes three or more points disposed along the 3D shape, and
the three or more points are equidistant from the reference plane.

19. The method according to claim 16, wherein:
the system includes a guide having a lumen extending along a straight section of the guide,
in use, the optical fiber is inserted within the lumen, and
the method further includes calibrating the optical fiber in accordance with the straight section.

20. The method according to claim 19, further comprising inserting the guide into the patient body.

21. The method according to claim 17, further comprising:
orienting the reference plane with respect to the reference frame so that a front view image of the 3D shape according to the reference plane is aligned with a front view according to the reference frame.

22. The method according to claim 17, further comprising fixing the reference plane with respect to the reference frame.

23. The method according to claim 17, further comprising:
comparing a curved portion of the 3D shape with a curved shape stored in memory of the system; and
as a result of the comparison, identifying the three or more points from the curved portion to define the reference plane when the curved portion of the 3D shape is consistent with the curved shape stored in memory.

24. The method according to claim 23, wherein in use, the curved portion of the 3D shape is disposed along a basilic vein, a subclavian vein, an innominate vein, or a superior vena cava, of the patient.

25. The method according to claim 23, wherein the system includes a plurality of curved shapes stored in memory, the plurality of curved shapes pertaining to a plurality of different insertion sites, wherein the method further comprises:
receiving input from a clinician defining an insertion site for the medical device;
selecting the curved shape from the plurality of curved shapes, the selected curved shape pertaining to the defined insertion site; and
comparing the curved portion of the 3D shape with the selected curved shape.

26. The method according to claim 25, wherein the input further defines the insertion site as located on a right side or a left side of the patient.

27. The method according to claim 23, wherein:
the system further includes a reference guide coupled with the medical device, and
the curved portion of the 3D shape is disposed along a pathway of the reference guide.

28. The method according to claim 27, wherein an orientation of the reference guide with respect to the patient defines an orientation of the 3D shape with respect to the patient.

29. The method according to claim 28, further comprising:
displacing the reference guide between a first guide orientation and a second guide orientation with respect to the patient to move the 3D shape between a first 3D shape orientation and a second 3D shape orientation with respect to the patient.

30. The method according to claim 16, wherein the system is coupled to a patient imaging system, the method further comprising:
receiving image data from the patient imaging system, and
rendering an image of the patient on the display along with the image of the 3D shape.
